(19) United States
(12) Patent Application Publication
(10) Pub. No.: US 2013/0053952 A1
(43) Pub. Date: Feb. 28, 2013

(54) CORNEA RESHAPING

(71) Applicants: Albert S. Jun, Baltimore, MD (US); Jennifer Elisseeff, Baltimore, MD (US)

(72) Inventors: Albert S. Jun, Baltimore, MD (US); Jennifer Elisseeff, Baltimore, MD (US)

(73) Assignee: THE JOHNS HOPKINS UNIVERSITY, Baltimore, MD (US)

(21) Appl. No.: 13/394,173

(22) PCT Filed: Sep. 7, 2010
(86) PCT No.: PCT/US10/47989

§ 371 Date: Mar. 5, 2012

Related U.S. Application Data
(60) Provisional application No. 61/239,918, filed on Sep. 4, 2009.

(51) Int. Cl. A61F 9/008; A61F 9/007; A61F 2/14

(52) U.S. Cl. 623/5.11

(57) ABSTRACT

A corneal reshaping kit may include a mold defining a concavity that is sized and shaped for receiving an outer corneal surface, such that a corneal surface pressed against the concavity will adopt a convex shape complementary to the concavity, and a biocompatible, flowable material suitable for introduction into a space within a cornea.

### CROSS-REFERENCE TO RELATED APPLICATION

This application claims the benefit of U.S. provisional application Ser. No. 61/239,918, filed Sep. 4, 2009, which is hereby incorporated herein by reference.

### BACKGROUND

Keratoconus is a disorder characterized by progressive thinning and steepening of the cornea with resulting loss of vision. This condition usually begins in the second decade of life and occurs in approximately 1 in 2000 individuals in the U.S. It is the most common indication for corneal transplantation in the U.S. and other developed countries. A similar condition called post-refractive surgery corneal ectasia, which also involves progressive thinning and steepening of the cornea, occurs as a complication of laser refractive corneal surgery. This condition is rarer than keratoconus although the clinical, diagnostic, and treatment features of post-refractive surgery ectasia are very similar to those of keratoconus.

Treatment for keratoconus involves spectacles and contact lenses in the early disease stages to correct the refractive errors induced by alterations in corneal shape. Eventually, the cornea becomes so misshapen that refractive correction is insufficient to provide adequate improvement in visual acuity. At this point, corneal transplantation is usually indicated to replace the diseased cornea with a cadaveric, normal donor cornea. In a limited number of keratoconus cases, intracorneal ring segments can be used to produce modest improvements in corneal shape in keratoconus patients in order to allow adequate vision correction with spectacle or contact lens use. Use of intracorneal ring segments is generally not regarded as a permanent treatment for keratoconus. At present, there is no FDA approved treatment to prevent the progression of keratoconus.

### SUMMARY

A corneal reshaping kit may include a mold defining a concavity that is sized and shaped for receiving an outer corneal surface, such that a corneal surface pressed against the concavity will adopt a convex shape complementary to the concavity, and a biocompatible, flowable material suitable for introduction into a space within a cornea.

A method of reshaping a cornea may include cutting a space in the cornea, injecting the flowable material into the space, pressing the concavity of the mold to an outer corneal surface of the cornea so that the outer corneal surface adopts a convex shape complementary to the concavity of the mold, and removing the mold from the outer corneal surface after the material has sufficiently hardened so that the outer corneal surface maintains the convex shape in the absence of the mold.

### BRIEF DESCRIPTION OF THE DRAWINGS

FIG. 1 depicts a cross-section of a cornea with keratoconus including a pocket cut in the cornea for insertion of an implant.

FIG. 2 depicts a solid corneal reshaping device.

FIG. 3 depicts insertion of a solid device into a pocket cut in the cornea.

FIG. 4 depicts a reshaped cornea with an implanted solid device, in cross-section.

FIG. 5 depicts a face-on view of a reshaped cornea with an implanted solid device.

FIG. 6 depicts insertion of a flowable material into a pocket cut in the cornea.

FIG. 7 depicts application of a mold to a cornea with implanted flowable material.

### DETAILED DESCRIPTION

Keratoconus and other disorders (especially refractive disorders) can be treated using the presently disclosed devices and methods for reshaping the cornea. A space is created within the cornea to receive a solid, semisolid, or liquid shaping agent. The agent may then be cured, if necessary, to allow it to maintain the desired shape. An external mold may be applied to the cornea to assist in achieving the desired shape.

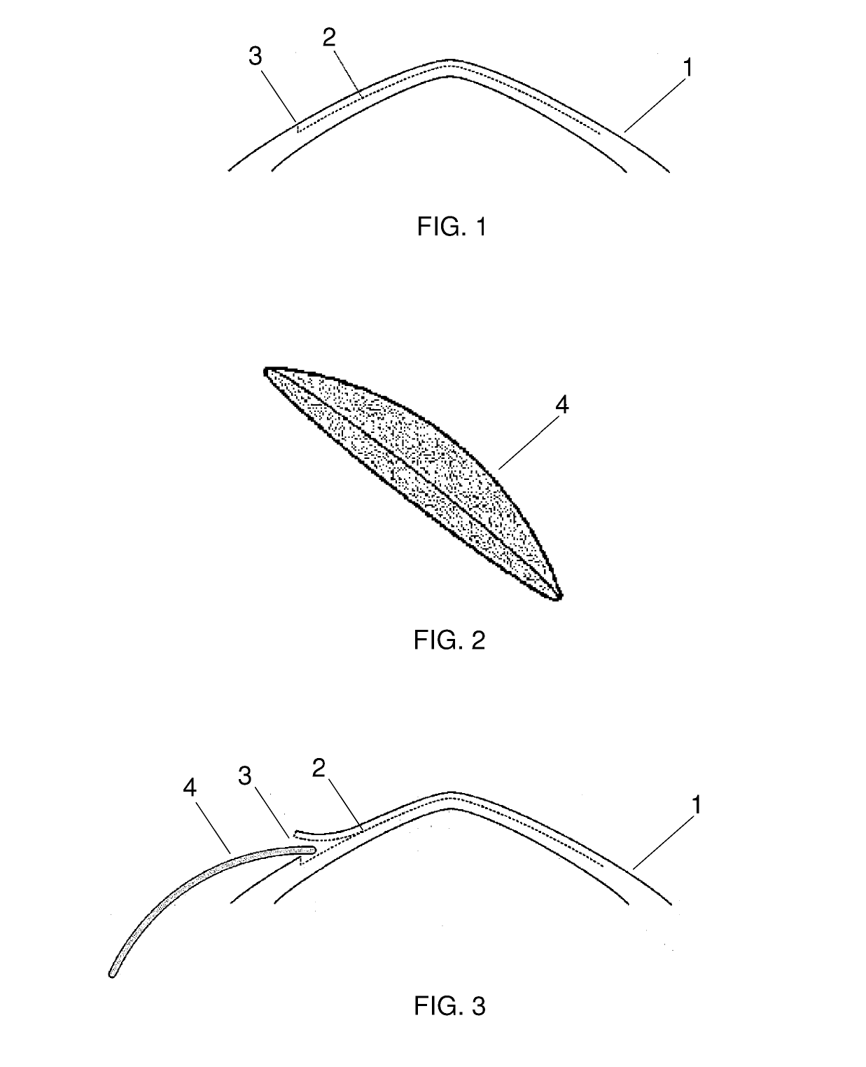

This basic approach may be implemented in a number of ways. For example, a femtosecond laser may be used to separate the central substance of the cornea (the stroma) to create a space which can accommodate the device. FIG. 1 depicts a keratoconus cornea 1 in which a space 2 has been created. Access incision 3 may also be created, to connect the internal space to the external corneal surface. Once the space and access insertion are created, the shaping agent can be introduced into the space, and the access incision can be secured, such as with standard corneal sutures, corneal adhesive, or other closure forms.

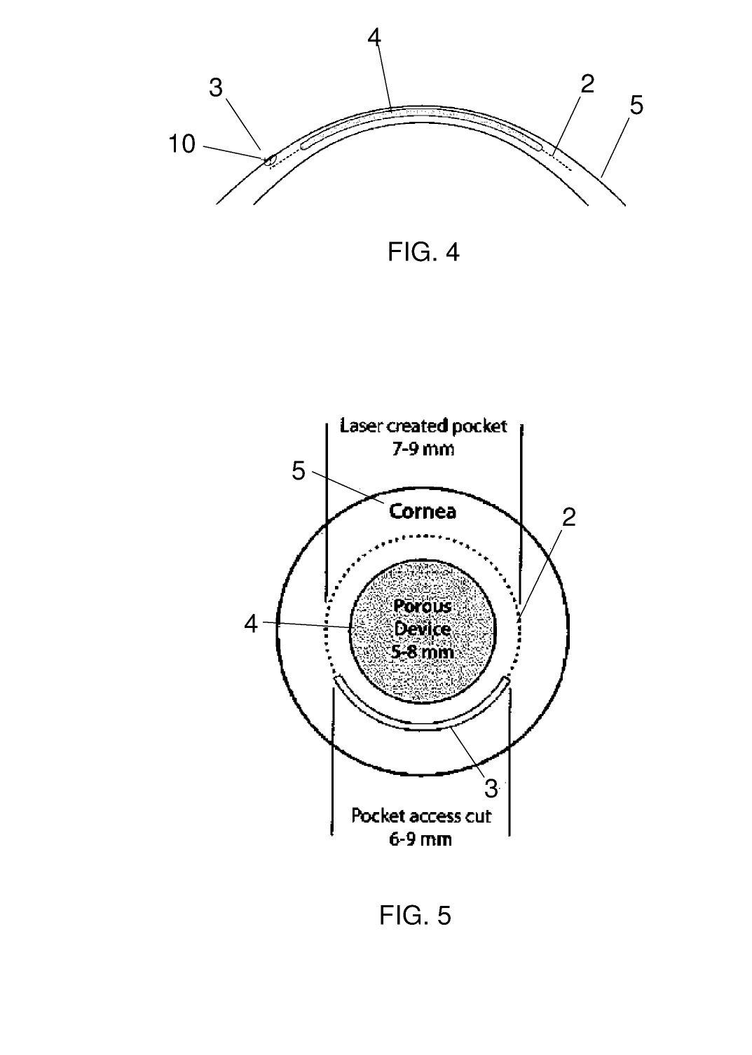

A variety of techniques may be used to form the space. Femtosecond laser technology is an attractive option because it is currently in widespread use in laser refractive surgery to precisely construct a separation in the anterior portion of the cornea prior to the reshaping of the cornea with the excimer laser to achieve vision correction. In one embodiment, the shaping agent is an implanted device 4, such as shown in FIG. 2. It may be an optically clear, thin, rigid, material with curved front and back surfaces which will fit within the corneal stromal space. FIG. 3 depicts the act of implanting the device 4 in the space 2 through the access incision 3. The dimensions, rigidity, and front and back curvatures of the device will be appropriate to modify and correct the shape of the subject's cornea to a degree sufficient to allow adequate visual improvement with or without concurrent use of spectacles or contact lenses. Additional features of the device will include long-term biological compatibility and stability of shape, rigidity, and clarity. FIG. 4 depicts the reshaped cornea 5 after the device 4 has been implanted, with the access incision 3 closed with corneal sutures 10.

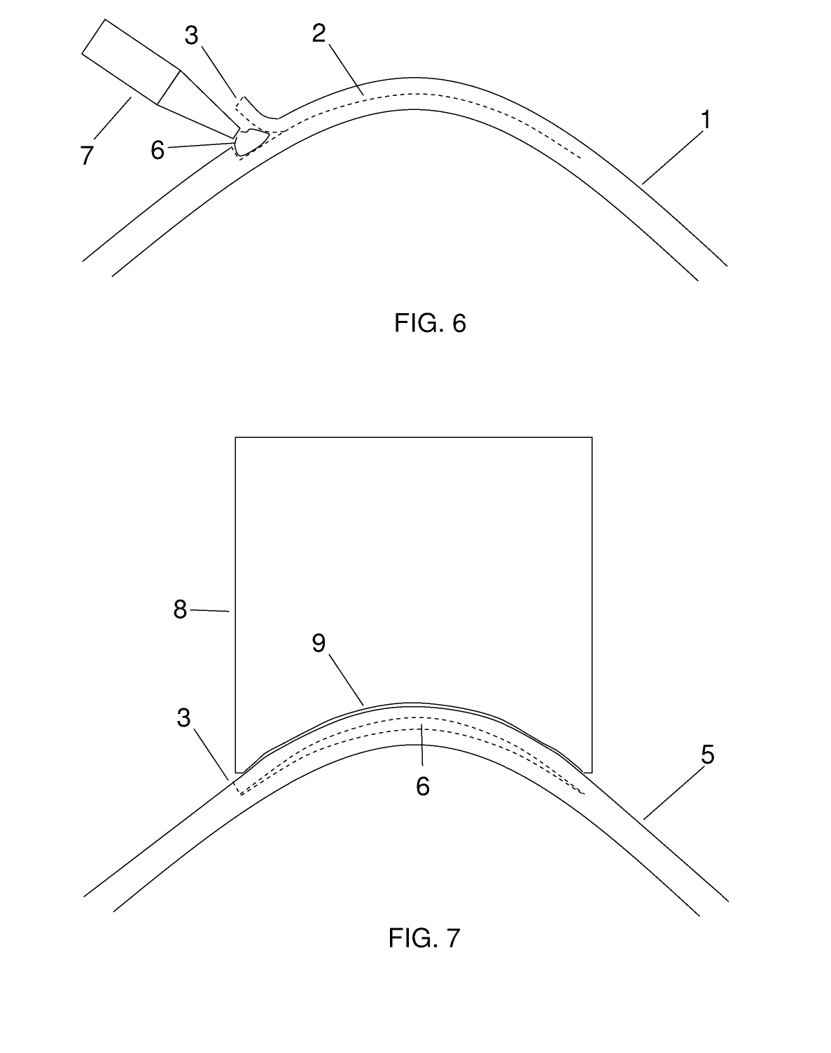

A variety of dimensions and properties are envisioned for the implanted device, including but not limited to the following, in which all numbers recited represent approximate values. Its physical dimensions may be between 5-8 mm in diameter. The device may be round with an anterior base curve between 8-9 mm and a posterior base curve between 6-9 mm. The thickness of the device may be between 50-150 micrometers. The device may include full-thickness fenestrations or pores created as part of the manufacturing process. These pores may be small (approximately 100 nm in diameter) or larger (1 mm or greater in diameter). The distribution of the pores may be non-uniform such that they may be more numerous and dense in certain areas of the device, such as the periphery, compared to others, such as the center. In addition (or alternatively), the device may define a central, circular aperture with a diameter up to 3 mm. In this case, the device would resemble an annulus with an inner diameter up to 3 mm and an outer diameter up to 8 mm. The device thereby leaves the visual axis unobscured. Some of these possible design features are shown in FIG. 5 in a front view of the reshaped cornea 5 with the device 4 implanted.

The implanted device may include an acrylic polymer with suitable biocompatibility to maintain corneal clarity and function.

The shaping agent could instead be a flowable material, such as a polymer. It may be injected into the space and then induced or allowed to harden. It may be introduced in a liquid, semisolid, or otherwise flowable form, for instance as a liquid, into the corneal stroma and induced or allowed to harden after placement. FIG. 6, for example, shows a cornea 1 that has been incised to form a space 2 with an access incision 3. Flowable material 6 is injected from an applicator 7 through the access incision 3 into the space 2. A particular preferred polymer for this application is polyethylene glycol, although other polymers or non-polymer biocompatible materials may be used. Examples of synthetic, biocompatible polymers are polyethylene glycol (PEG), polyvinyl alcohol (PVA) and block copolymers, such as the Pluronic® compounds, as explained in U.S. Pat. Pub. 2010/0010187 A1, which is herein incorporated by reference. It is preferred that the material be transparent. FIG. 7 shows the cornea 5 after the material 6 has been injected into the space 2 (not shown).

An external shape mold 8 may be applied to the external corneal surface during the time in which the material 6 hardens, in order to influence the shape that material 6 will take. The mold will impose a desired shape on the external surface of the cornea. The corneal stroma and the space formed in it will consequently be changed in shape, and the flowable material will conform to the shape of the space. Once the material hardens, its rigidity will maintain the shape imposed on the cornea, including the external corneal surface, without the external mold present. The external mold is typically applied during the time after the flowable material has been injected into the cornea and before the material becomes rigid. The mold may be applied with compression to help impress on the cornea a shape complementary to the mold. The surface of the mold 9 in contact with the corneal surface may have a shape approximating normal corneal curvature (e.g., for cases in which the cornea is abnormally shaped such as keratoconus or post-refractive surgery corneal ectasia) or a shape designed to compensate for refractive errors elsewhere in the cornea or eye (e.g. myopia, hyperopia, presbyopia, and/or astigmatism). Application of the mold to the external corneal surface while the liquid polymer solidifies will impart rigidity and shape to the implant which will maintain the cornea with a flatter, more regular shape to improve the refractive error and poor vision resulting from keratoconus. The mold may be removed at any time once the injected material is capable of retaining the desired shape.

The flowable shaping agent may be or include a hydrogel which may be produced using a crosslinking agent solution.

The flowable shaping agent may be or include a hydrogel produced through exposure to light of a specified wavelength. In such a light activated liquid polymer, the appropriate light source will be applied to the polymer within the cornea by shining it on the shaping agent, for example, through the mold while the mold is applied to the external corneal surface. If the light is applied in this manner, the mold should be made of material(s) that transmit at least the portion of the light required for activation. Typical suitable materials include polyurethane and polycarbonate. The mold can be formed with one of more conduits of material that transmit the required light. The conduits may also be empty spaces. The mold and light source if applicable will be applied to the external corneal surface for a time period sufficient to allow the liquid polymer to become fully rigid.

A flowable shaping agent, such as a polymer, may be cured in any number of ways, including application of light, application of heat, application of particle radiation, or use of a chemical additive. A photoinitiator may be included in the flowable agent. Examples of photoinitiators include benzophenone, trimethylbenzophenone, thioxanthone, 2-chlorothioxanthone, 9,10-anthraquinone, bis-4,4-dimethylaminobenzophenone, benzoin ethers, benzilketals, α-dialkoxyacetophenones, α-hydroxyalkylphenones, α-amino alkylphenones, acylphosphine oxides, benzophenones/amines, thioxanthones/amines, titanocenes, 2,2-dimethoxy acetophenone, 1-hydroxycyclohexyl phenyl ketone, 2-methyl-1-[4-(methylthio)phenyl]-2-(4-morpholinyl)-1-propan one, 2-hydroxy-1-[4-(hydroxyethoxy)phenyl]-2-methyl-1-propanone, α-hydroxy-ketones and benzilidimethyl-ketals, e.g. Irgacure 651 and 184, and Darocur 1173, marketed by Ciba Chemicals, Rose Bengal, camphorquinone, erythrosine, and mixtures thereof, and so on, as explained in U.S. PGPub. 2009/0074868 A1, which is incorporated herein by reference. Ethyl eosin (a tertiary amine and dye), in combination with N-vinyl pyrrolidone (accelerator) can act as a photoinitiator in the presence of visible green light.

If a chemical additive is used to harden the polymer, it may be applied either before or after the polymer has been injected. Examples of chemical additives to cause curing include those disclosed in U.S. PGPub No. 2010/0137241 A1. The flowable shaping agent may include a thermoresponsive material, such that it cures when exposed to heat, such as body heat. Examples of thermal initiators that form free radicals at moderate temperatures, include benzoyl peroxide, with or without triethanolamine, potassium persulfate, with or without tetramethylethylenediamine, and ammonium persulfate with sodium bisulfite, as explained in U.S. PGPub. 2009/0324722 A1, which is incorporated by herein by reference. The curing may involve any process by which the injected material goes from a flowable state to a state in which it is capable of maintaining a desired shape. This could involve, for example, crosslinking, polymerization or a phase transition.

The flowable shaping agent may also include (or have added to it after injection) one or more materials that promote tissue growth, to stimulate repair and/or reshaping, such as cells, stem cells, and bioactive molecules that promote growth, recruitment, differentiation, vascularization, etc.

In another embodiment, the cornea may be reshaped using a flowable material but without the use of an external mold. As in some other embodiments, a space within the corneal stroma is created, possibly by use of a femtosecond laser. A flowable material, such as a curable polymer, is then injected into the space in the corneal stroma. While the material is being injected the shape of the cornea is monitored. When a desired shape is achieved, the injection of material ceases, and the injected material is hardened or allowed to harden such that the desired shape is maintained. In this way it is possible to achieve a desired shape of the cornea by injecting the material but without applying an external mold.

The cornea may also be reshaped with a combination of a rigid piece such as an implanted device described above and a flowable material.

Methods and devices for modifying the shape of a cornea may be applicable to treatment of keratoconus and other conditions, for example myopia, hyperopia, presbyopia, and astigmatism.

### EXAMPLE

Corneal Reshaping using External Mold and Flowable Shaping Agent

Explanted pig eyes were used to test the feasibility of the method. External molds of various shapes were prepared from polyurethane or polycarbonate. Intrasomal corneal pockets were formed in the eyes using a femtosecond laser. Light-activated flowable material (polyethylene glycol diacrylate, with ethyl eosin and N-vinyl pyrrolidone for photoinitiation) was injected to the pockets thus created. An external mold was pressed against the external surface of an injected cornea. While the mold was held in place, visible green light was applied. Results of keratometry measurements taken before and after applanation follow.

| | Mean Keratometry | Mean Keratometry | |
|---|---|---|---|
| | (Diopters) Before | (Diopters) | Keratometry |
| | Applanation | After Applanation | Difference |
| Eye | Avg. 3 measurements | Avg. 3 measurements | (Diopters) |
| 1 | 46.3 | 54.4 | +8.1 D |
| 2 | 38.4 | 46.6 | +8.2 D |
| | | | Mean ± SD |
| | | | Difference |
| | | | +8.2 ± 0.1 D |

Any discussion of the related art throughout the specification should in no way be considered as an admission that such art is widely known or forms part of general knowledge in the field.

1. A corneal reshaping kit comprising:
a mold defining a concavity that is sized and shaped for receiving an outer corneal surface, such that a corneal surface pressed against the concavity will adopt a convex shape complementary to the concavity; and
a biocompatible, flowable material suitable for introduction into a space within a cornea.

2. The kit of claim 1 wherein the flowable material comprises a polymer.

3. The kit of claim 2 wherein the polymer comprises a polyethylene glycol.

4. The kit of claim 2 wherein the polymer is curable.

5. The kit of claim 4 wherein the polymer is curable by application of light.

6. The kit of claim 5 wherein the flowable material comprises a light sensitive curing activator.

7. The kit of claim 4 wherein the polymer is curable by the application of heat.

8. The kit of claim 4 wherein the polymer is curable by application of a chemical additive.

9. The kit of claim 1 wherein the material is transparent.

10. The kit of claim 1 wherein the concavity is sized and shaped to correct keratoconus.

11. The kit of claim 1 wherein the concavity is sized and shaped to correct myopia, hyperopia, presbyopia, or astigmatism.

12. The kit of claim 1 wherein the flowable material comprises a hydrogel.

13. A method of reshaping a cornea with the kit of claim 1 comprising:
cutting the cornea to create a space defined by the cornea;
injecting the material of the kit of claim 1 into the space;
pressing the concavity of the mold of the kit of claim 1 to an outer surface of the cornea so that the outer corneal surface adopts a convex shape complementary to the concavity of the mold;
removing the mold from the outer corneal surface after the material has sufficiently hardened so that the outer corneal surface maintains the convex shape in the absence of the mold.

14. The method of claim 13 wherein cutting the cornea to create the space comprises using a laser.

15. A method of reshaping a cornea comprising:
cutting an annular pocket in the cornea;
injecting a biocompatible, flowable material into the pocket;
monitoring the shape of the cornea while the material is being injected;
ceasing injection of the material when a desired corneal shape has been achieved; and
hardening the material or allowing the material to harden, such that the cornea maintains the desired corneal shape.

16. The method of claim 15 wherein cutting the annular pocket comprises using a laser.

17. A method of reshaping a cornea comprising:
creating a space in the cornea by separating the corneal stroma;
injecting a liquid polymer into the space created by separating the corneal stroma;
applying a mold to an external corneal surface so as to impart a desired shape to the cornea; and
inducing the polymer to harden so that the cornea maintains the desired shape.
